(19) United States
(12) Patent Application Publication
(10) Pub. No.: US 2011/0295284 A1
(43) Pub. Date: Dec. 1, 2011

(54) SURGICAL MESH AND METHOD OF MANUFACTURE

(75) Inventors: Dan Purdue, Henley-In-Arden, Worcestershire (GB); Claire Fenlon, Rugby (GB)

(73) Assignee: MANDACO 569 LIMITED, REDDITCH WORCESTERSHIRE (GB)

(21) Appl. No.: 13/148,249

(22) PCT Filed: Feb. 5, 2010
(86) PCT No.: PCT/GB10/50190

§ 371 Date: Aug. 5, 2011

(30) Foreign Application Priority Data
Feb. 5, 2009 (GB) 0901779.9

(51) Int. Cl. A61B 17/00; B23P 11/00

(52) U.S. Cl. 606/151; 29/428

(57) ABSTRACT

The present invention provides a surgical mesh device (110) suitable for use in reinforcing or supplementing damaged ligaments while healing takes place or for use in conjunction with autolgous grafts for reconstructing torn ligaments. The surgical mesh includes a non-load bearing radiopaque marker (120, 122) enabling in situ imaging of the surgical mesh immediately following surgery it and subsequent changes in the surgical mesh over time.

The present invention relates to a surgical mesh and a method of manufacturing such devices and their use. These surgical mesh devices may be used for reinforcing or supplementing damaged ligaments while healing takes place or for use in conjunction with autologous grafts for reconstructing torn ligaments. The present invention relates particularly but not exclusively to such devices when made at least partially opaque to X-ray energy, thereby to make them visible during medical imaging procedures.

EP 437,174A discloses an artificial ligament device and describes also the corresponding method of its manufacture comprising securing a plurality of tows of biocompatible material side-by-side in a flat elongate array, bending the tows back at one end of the device to form a bent portion with the tows grouped together round the bent portion.

EP 693,909A (Surgicraft) discloses artificial ligament devices having a strong integral eye at one or both ends for attachment of a pulling cord or wire and/or for fitting round an anchor member for securing to a bone.

Whilst the above patent applications describe perfectly acceptable surgical mesh devices they suffer from the same problem in as much as it is difficult if not impossible to view such a device under X-ray imaging as the material used has a radio-absorbency substantially the same as the surrounding soft tissue.

The property of blocking X-rays generally proves to be more effective the higher the density of the material they pass through. For this reason, lead is used extensively in the realisation of all shielding apparatus both in medicine and in the industrial sector but lead is not an acceptable material for permanent or semi-permanent implantation into the human or animal body. Other dense materials such as titanium, stainless steel and the like are also known for use in the body and are both bio-compatible and opaque to X-rays and special ceramics have been used to produce features that must be detected using X-ray analysis techniques. Whilst such materials and their properties are known in the medical field they do not readily lend themselves to the manufacture of high tensile load capacity medical products such, as surgical meshes and the like. It is therefore an object of the present invention to provide a design of surgical mesh device or the like which overcomes the problems associated with the prior art whilst also providing features which are not transparent to X-rays, which will make the device visible under X-ray imaging whilst retaining the required degree of flexibility and tensile strength that such devices require in order to perform their correct bio-mechanical function.

The use of radiopaque material in artificial ligaments is known from the prior art for general imaging of the ligament once in situ. For example, U.S. Pat. No. 4,883,486 discloses an artificial ligament made up of a core of elongate filaments surrounded by a tubular sheath. To assist in X-ray viewing of the device after it has been implanted, a barium sulfate filled polypropylene filament may be included within the core. FR 2,722,976 discloses an artificial ligament, made of a narrow strip of knitted material which includes a single line of radiopaque material running across the ligament for visualisation of the ligament after implant. Other documents, such as GB 1,317,417, disclose including radiopaque fillers in an artificial tendon or, such as EP 561,710A, disclose impregnating the ligament fibres with radiopaque agents.

Post operatively, patients may represent with pain at or close to the site at which a surgical mesh device has been used. Diagnosis of the cause of that pain can be difficult, especially given that it is undesirable to re-open the operative site in order to carry out a visual inspection. One possible cause of the further pain may be to do with changes to the implanted device.

Accordingly, in a first aspect the present invention relates to a surgical mesh comprising an elongate length of load bearing fibres and additionally comprising a non-load bearing radiopaque material anchored at more than one location along the length of the mesh, the radiopaque material enabling in situ imaging of the surgical mesh immediately following surgery during which it has been implanted and imaging of subsequent changes in the surgical mesh over time.

Hence the current invention provides a surgical mesh which, once implanted into a patient, can be imaged to show changes in the morphology of that mesh in situ without requiring the operative site to be re-opened for visual inspection. The prior art proposals do not give this option.

In most of the prior art discussed above, the radiopaque filaments are load bearing. Although this will enable medical staff to check the location of the ligament or tendon when inserted, it will not provide such further information about the implant as is possible from the present invention.

In FR 2,722,976, the radiopaque filament is not load-bearing. However, it does not extend along any part of the length of the artificial ligament and does not assist in understanding post-operative changes to the implant.

In one embodiment, the radiopaque material runs along at least part of the length of the surgical mesh device, such radiopaque material being anchored to the mesh at two or more points along the length axis of the mesh and the length of radiopaque material between such anchor points being longer in length than the distance of mesh between which the radiopaque material is anchored and wherein the length of radiopaque material between the or each pair of anchor points is sufficiently greater than the distance between the anchor points that, immediately following insertion, the radiopaque material will be non-load bearing, but whereby at or immediately following any rupture of the surgical mesh, it will become load bearing and will itself break. This facilitates visualisation as to where the surgical mesh breaks, but without risking the radiopaque material rupturing before the surgical mesh and giving a mis-diagnosis.

There are number of ways in which the radiopaque material may be arranged in relation to the mesh. The radiopaque material may run along only a short part of the mesh or it may run along the full length. Where the radiopaque material is anchored at either side of potential rupture points, then this will provide information about the location at which any subsequent rupture occurred. The information about the location of the rupture point will be made more accurate where the radiopaque material is anchored at intervals along the length of the mesh. By anchoring the radiopaque filaments at intervals along the surgical mesh, any breakage in the filaments will be more closely aligned to the site of rupture of the mesh. An X-ray image of the area will show the radiopaque filaments on each side of the mesh rupture point. By comparing the different lengths of those filaments, medical staff will be able to identify the location of any breakage and so be better informed in the planning further treatment.

An alternative to anchoring a long radiopaque filament along at intervals along the surgical mesh is to anchor multiple discrete lengths of radiopaque material. These can then be arranged to extend the full length of the surgical mesh or be restricted only to certain parts of the mesh.

Optionally, radiopaque material may also be anchored on the surgical mesh in discrete locations arranged at predetermined distances along a least part of the length of the surgical mesh device. These perform the function of enabling detection as to whether or not the surgical mesh has stretched. An X-ray of the site is taken during or immediately after the implant operation. When the patient represents with pain, a second X-ray is taken of the same site. Ideally, those images should be taken from the same angle and to the same scale. Where the mesh has stretched in the region of the radiopaque material, the distance between the discrete locations will have increased. The radiopaque material may be arranged as 4 to 10 discrete anchor points in, for example, two parallel rows over a distance of say 2 to 5 mm. Preferably, changes in surgical mesh length of at least 15% will be detectable. More preferably changes in surgical mesh length of at least 10% will be detectable.

Where slack radiopaque material is anchored to a surgical mesh device and inserted into a patient, there is a risk that movement of the surgical mesh in situ could result in the radiopaque material wearing either against the surgical mesh or against the patient's bones. This could cause damage to the surgical mesh or to the patient. So when designing a surgical mesh device to fit a particular location, the radiopaque material is preferably located on the surgical mesh where such risk of damage is minimised. For example, where a surgical mesh is intended to be attached in a “C” shape between two bones lying in different planes, it will have a first side which will contact bone and a second side which will not. The radiopaque material is preferably anchored to the second side. Alternatively, where a surgical mesh is intended to be attached in an “S” shape between two bones lying in different planes, it will have a first end which will contact bone on a first side and a second end which will contact bone on the second side. In this case, the radiopaque material is preferably anchored to the second side at the first end and to the first side at the second end.

With a “C”, “S” or like configuration, there will be a section of the surgical mesh which spans the gap between the bones. In such surgical mesh devices, radiopaque material may be located in that intermediate section of the surgical mesh which does not contact bone

Where the surgical mesh is designed to pass through a tunnel in bone, then its entire surface may contact bone. In such arrangements, the surgical mesh may be in the form of a sleeve, with the radiopaque material located within that sleeve.

In a second aspect, the present invention provides a surgical mesh device comprising a plurality of tows of biocompatible or bioabsorbable material secured side-by-side in a flat elongate array and being looping to form an eye at at least one end of the device with the tows grouped together round the eye. Suitably as least one eye is strengthened for attachment to an anchor point. This may be done by whipping the tows together and optionally also lashing the tows at the base of the eye. Preferably radiopaque material is coupled at or close to the eye. Alternatively, the surgical mesh may have eyes at both ends, with the radiopaque material being coupled at or close to one or both eyes. The surgical mesh of the second aspect may form part of the surgical mesh device of the first aspect.

Each tow may be formed of a mutiplicity of polyester (or similar biocompatible or bioabsorbable) filaments and the tows secured together by light braiding, to form the flat array. The array (or parts thereof) may be treated with a biocompatible, bioabsorbable or pharmacologically active coating. Preferably, the surgical mesh will support and encourage host tissue growth to assist in healing. An open mesh is particularly suitable for encouraging such growth.

The surgical mesh device may be in the form of a sleeve. This may be formed from a sufficient width of an initial flat array of tows, or several lengths of such arrays may be sewn together edge-to-edge. The extreme edges may then be mated together and secured to each other to form a sleeve, for example by stitching. Where a radiopaque filament is incorporated within the sleeve, it will be anchored at two or more points inside the sleeve, the length of radiopaque material between the or each pair of anchor points being longer in length than the distance between the points to which the radiopaque material is anchored. This ensures that it is not load bearing.

According to a third aspect of the invention, there is provided an artificial ligament or a ligament augmentation device comprising the surgical mesh device according the first and/or second aspect of the invention. The surgical mesh device of this invention can be used as an artificial ligament or a ligament augmentation device. It may also serve as a delivery device for an autologous graft. The autologous graft could be wrapped within a sleeve surgical mesh device, or may be secured to one end of the surgical mesh device.

According to a fourth aspect, the invention also comprises a surgical kit which includes a surgical mesh device according to the first and/or second aspect of the invention, together with means to anchor the device within the patient. The anchoring means may take the form of one or more anchoring members, such as a screw which can secure an eye of the surgical mesh device to a bone. Alternatively, the kit may include sutures for attaching the surgical mesh device to tissue or staples to attach it to another bone. Frequently, a surgical kit will include a set of surgical mesh devices of varying lengths and a measuring device. During surgery, the measuring device is used at the operative site to determine which length of surgical mesh device is required. Then the selected surgical mesh device is implanted and secured. Additionally or alternatively, the kit may comprise one or more tools for inserting and securing the surgical mesh device. In use, the surgical mesh may be affixed to a bone, such as the clavicle, by looping it around the coracoid bone and passing the first eye through the second eye to secure it in place.

The radiopaque material may be made of any metallic filament of medical grade or a medical grade polymer based yarn. Suitably this should be biocompatible and suitable for permanent implantation. A number of materials lend themselves to use as radiopaque filaments or markers in the surgical mesh and devices according to the present invention and include, but are not limited, to materials selected from the group consisting of tungsten carbide, tungsten boride, the metals platinum, tantalum, iridium, tungsten, rhenium titanium, silver and gold, stainless steel alloys of said metals, high density polymers and combinations thereof. Preferred examples include titanium alloy filaments, tantalum alloy filaments and polypropylene yarn loaded with a high level of barium sulphate.

The radiopaque material preferably constitutes between 0.5% and 2% by volume of the total volume of the device itself such as to retain a high degree of commonality with the required bio-mechanical properties of such devices. One or more of the components that make up the surgical mesh device may be replaced or supplemented by such a radiopaque material such as to make it visible under X-ray analysis. The radiopaque material may be added by hand after the surgical mesh has been formed or may be incorporated into the device during manufacture. When incorporated into the device during manufacture the radio-opaque material should not be tensioned in the same manner as the other components. It is the intention that the radiopaque material does not to experience tensile load during ordinary use, but is capable of becoming tensioned at or shortly after rupture of the surgical mesh, at which point it too should break. The tensile properties of the material should be selected accordingly, with the tensile strength of the radiopaque material suitably being of lower tensile strength than the surgical mesh as this is then likely to break quickly once the load which has already ruptured the surgical mesh becomes concentrated on the radiopaque material.

According to a fifth aspect, the invention comprises a method of making surgical devices according to the first and/or second aspect of the invention. First the surgical mesh is created. Then non-load bearing radiopaque material is anchored at one or more locations along the length of the mesh. This may be done by anchoring a length of radiopaque material to at least part of a surgical mesh, the length of radiopaque material being anchored to the surgical mesh at at least two locations along the length of the mesh and the amount of radiopaque material between points of anchoring being longer in length than the distance between which it is anchored.

Accordingly, a sixth aspect of the invention relates to the use of surgical mesh devices according to the first and/or second aspect of the invention in surgery and to the surgical methods of implanting such devices for example to replace or augment ruptured ligaments or tendons. During surgery one end of the surgical mesh device is secured to a bone. The surgical mesh device is then extended across the surgery site and the other end of the surgical mesh device is secured to another bone or soft tissue.

The surgical meshes of the present invention may be used to assist in the repair of acromioclavicular joint dislocation. The surgery comprises measuring the distance from the coracoid to the clavical in its reduced position, selecting a surgical mesh device which has a length substantially the same as the measured length, looping the mesh around the coracoid, passing a first eye through the second eye to secure a first end in place on the coracoid, attaching an anchor member to the clavicle and engaging the eye at the second end of the mesh to said anchor member,

A seventh aspect of the invention comprises steps in medical diagnosis of causes of pain in a post-operative patient into which a surgical mesh device of the present invention has been implanted, for example by X-raying the patient and identifying the radiopaque material of the surgical mesh device. This may include identifying rupture of the surgical mesh device and the location of such rupture or it may involve comparing post-operative X-ray images with new X-ray images to determine whether the surgical mesh has stretched.

Accordingly in the seventh aspect the invention provides a method of detecting changes in a surgical mesh which comprises inserting a surgical mesh device according to the first and/or second aspects of the invention, imaging the operative site post operation, re-imaging the site following the patient representing with pain and comparing the images. The insertion of the surgical mesh may includes the step(s) of securing the mesh to an anchor member at one or both ends. Where the surgical mesh device has an eye proximate each end, the insertion of the surgical mesh may include the step(s) of securing the mesh in the shoulder by looping it around the coracoid bone and passing the first eye through the second eye to secure a first end in place and securing the second end to an anchor member. The present invention also provides a method of detecting rupture in a surgical mesh device, including the steps of inserting a surgical mesh according to a first and/or second aspect of the invention in a patient and imaging the site when the patient represents with pain and reviewing the image

The present invention, its manufacture and uses will now be described, by way of example only, with reference to the accompanying drawings, in which:—

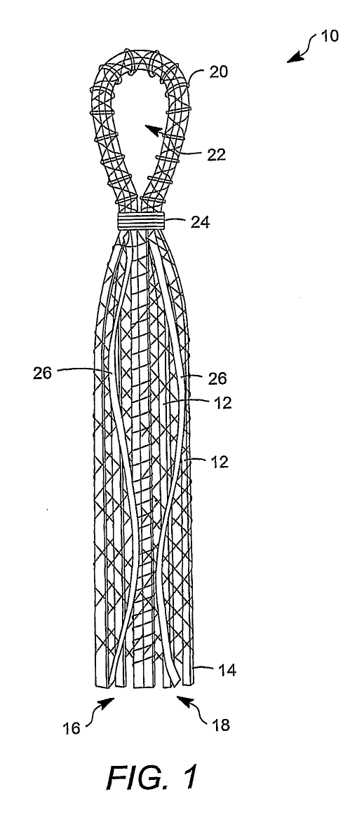

FIG. 1 shows a surgical mesh according to one embodiment of the invention;

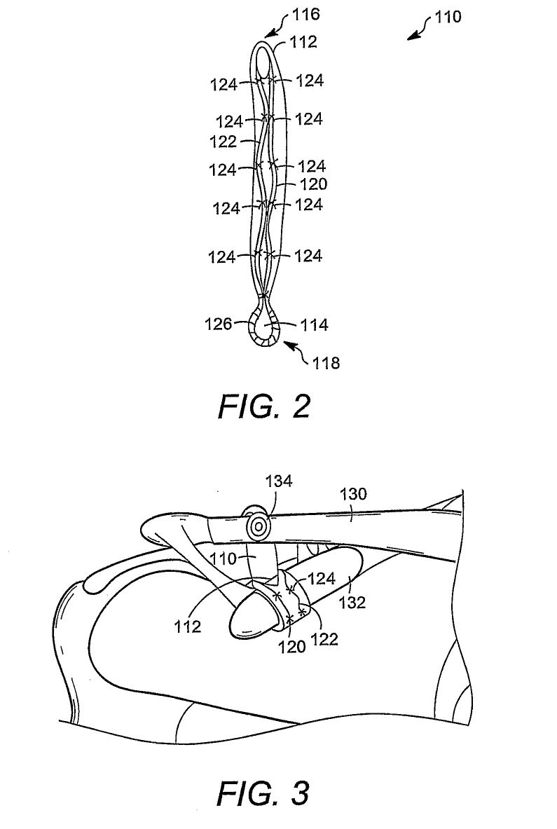

FIG. 2 shows a surgical mesh according to another embodiment of the invention;

FIG. 3 shows the surgical mesh of FIG. 2 in situ in a collar bone;

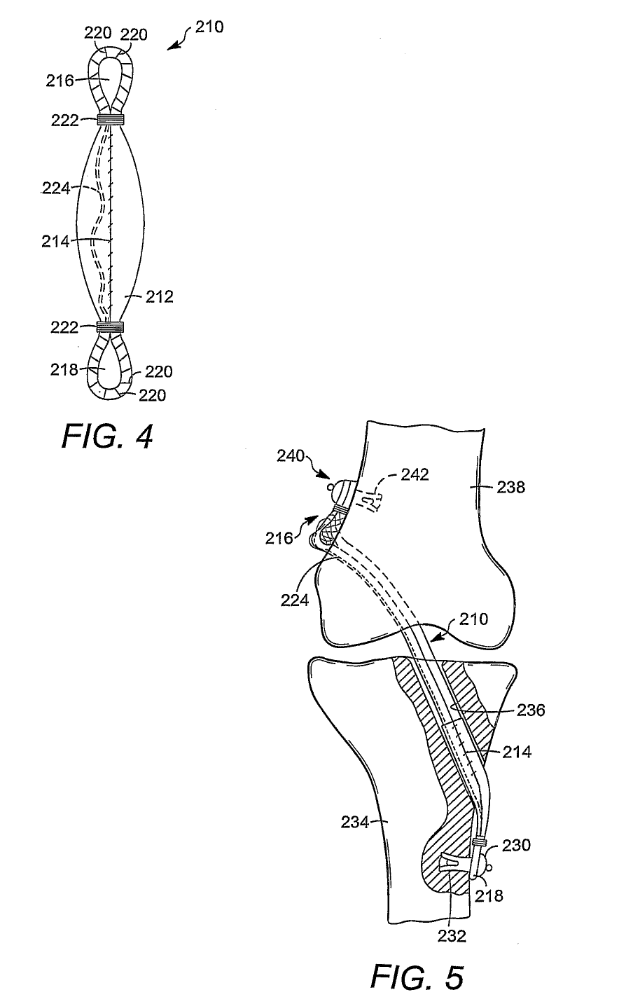

FIG. 4 shows a surgical mesh according to another embodiment of the invention;

FIG. 5 shows the surgical mesh of FIG. 4 in situ in a knee;

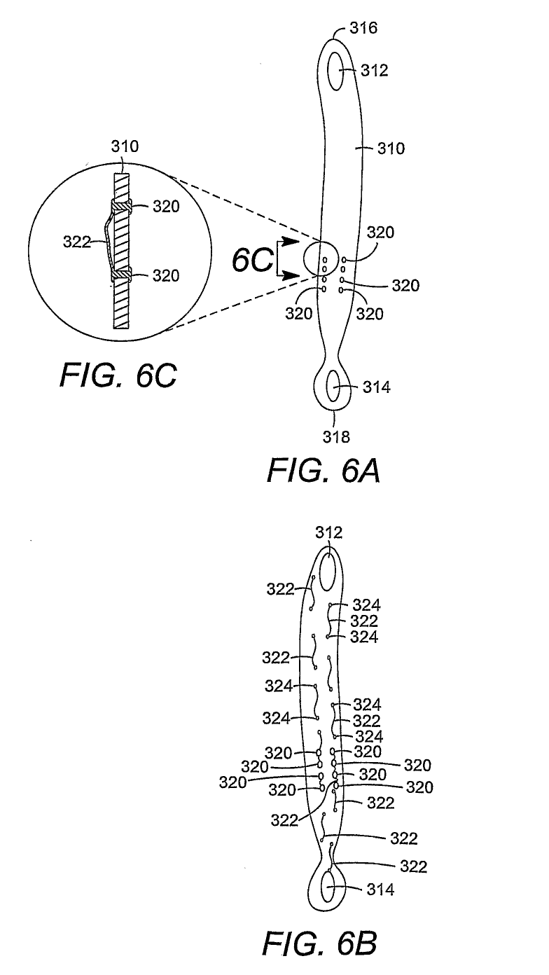

FIGS. 6a, b and c show a surgical mesh according to further embodiment of the invention;

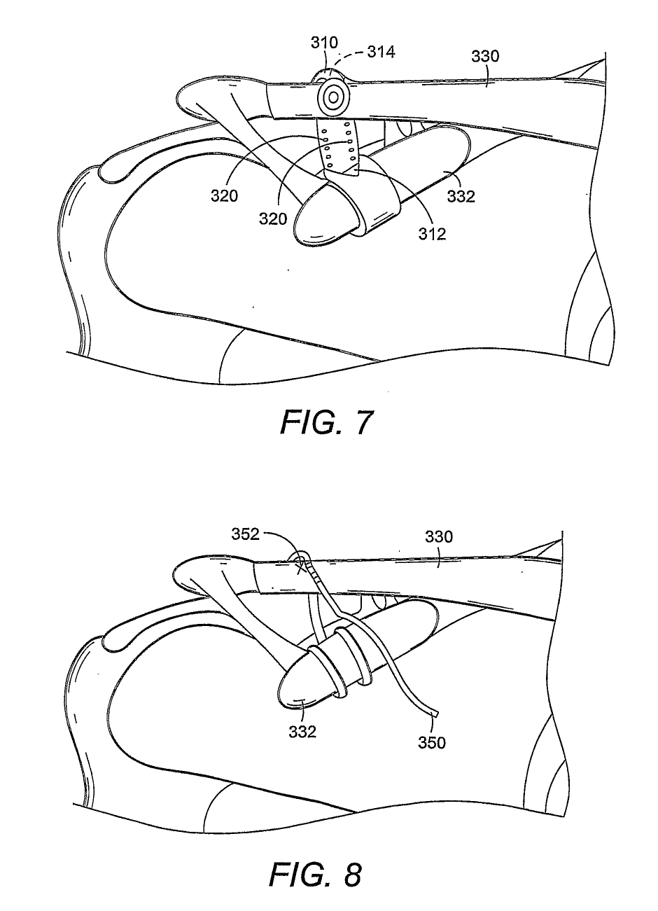

FIG. 7 shows the surgical mesh of FIG. 6 in situ in a collar bone;

FIGS. 8 to 10 illustrate stages in the repair of acromioclavicular joint disclocation; and

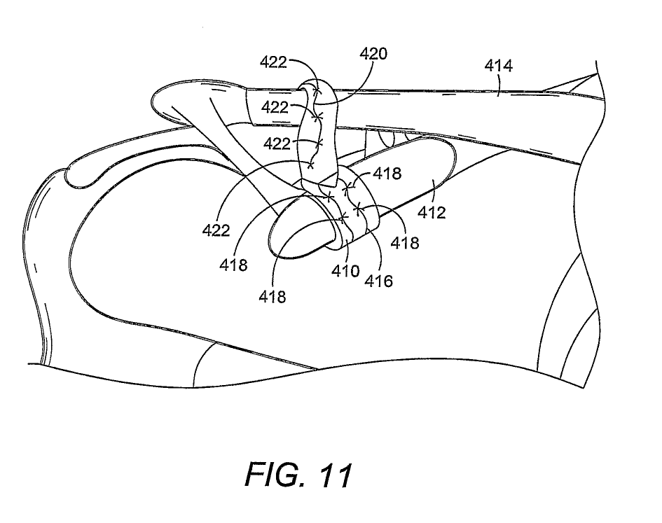

FIG. 11 shows an alternative attachment structure.

FIG. 1 shows a surgical mesh 10 which comprises a plurality of tows 12 of biocompatible material secured together by braiding 14 to form a flat elongate array. The tows may be made from polyester fibres. The tows are looped intermediate their ends 16, 18 and the central portion has whipping 20 to form an eye 22 at one end of the surgical mesh device which has a lashing 24 at the base of the eye. Radiopaque filaments 26 are held in connection with the eye 22 at the whipping 20 and lashing 22 and are also connected to the surgical mesh 10 at the other free ends 16, 18 by any convenient means, such as by being stitched in. However, the filaments 26 could also extend over only part of the length of the surgical mesh, for example, from the lashing 22 to a point half way along the mesh.

The filaments 26 are longer in length than the distance between the whipping 20 and their points of attachments. So when the surgical mesh is placed in situ within a patient and the tows within the mesh are stressed, the radiopaque filaments 26 will remain in a non-load bearing conformation.

FIG. 2 shows an alternative surgical mesh 110, which has an eye 112, 114 at each end 116, 118. One eye 114 has whipping or braiding 126. However, the other eye 112 does not. Two radiopaque filaments 120, 122 run from one eye 112 to the other 114. The filaments 120, 122 are longer than the distance between the two eyes 112, 114 and so are not load-bearing when the surgical mesh is inserted into the patient. In the embodiment shown in FIG. 2, the radiopaque filaments 120, 122 are secured at intervals to the surgical mesh. This is illustrated at points 124 along the length of the filaments. The filaments may be secured to the mesh by extra stitches or may be woven or braided into the mesh at that point. The filaments remain loose between each pair of adjacent attachment points 124, so as to ensure that, under normal usage, the radiopaque filaments remain non-load bearing at each section throughout their length.

In the embodiment shown in FIG. 1, a rupture of the surgical mesh between the lashing 24 and the ends 16, 18, will result in breakage at the weakest point along the radiopaque filaments 26. This will not necessarily be close the point along the length of the surgical mesh were the rupture occurred. The embodiment shown in FIG. 2, enables the point of rupture of the surgical mesh to be more accurately identified. A rupture of the surgical mesh between two adjacent anchor points 24 will result in breakage in the adjacent section of the radiopaque material.

FIG. 3 shows a surgical mesh of the type shown in FIG. 2 in situ in a shoulder to assist in the repair of an acromioclavicular joint dislocation. During the operation to reduce the clavicle 130, the surgical mesh 110 is wrapped around the coracoid bone 132. One eye 114 is passed through the second eye 112 to secure one end of the surgical mesh to the coracoid 132. As this eye 112 is not whipped or braided, the mesh remains soft and will lie closely against the coracoid. The surgical mesh is then threaded under and over the clavicle 130 and attached to the clavicle at a fastening means, such as a screw 134. The whipped eye 114 is fitted over the screw and the screw tightened to hold it in place. The radiopaque filaments 120, 122 remain untensioned around the outer surface of the mesh.

Once the surgical mesh has been implanted, its biocompatibility encourages ingrowth of host tissue. This has the effect of further stabilising and strengthening the mesh. Usage of the joint will place stress on the surgical mesh and it may lengthen. The risk of the mesh stretching will reduce over time as the tissue ingrowth increases.

Although any stretching of the surgical mesh will cause the looseness in the radiopaque filaments to decrease, the length of those filaments is selected to accommodate this and to ensure that they do not become tensioned whilst the surgical mesh remains in tact. Should the surgical mesh rupture, the radiopaque filament will become fully load bearing across the rupture site and then itself break. This will then be visible to X-ray.

As can be seen from FIG. 3, the radiopaque filament is located on the side of the surgical mesh away from the contact with the bone, so as to minimise the risk of the filament rubbing against the bone or between the bone and the fibres of the surgical mesh as the patient moves in his/her daily life. This in turn will reduce the risk of the radiopaque filament causing wear of the other fibres in the surgical mesh.

The device 210 shown in FIG. 4 comprises a sleeve of material 212, which is joined at a row of stitching 214. Eyes 216, 218 at each end are formed with whippings 220 and lashings 222. The sleeve 212 may also contain an autologous graft. In this embodiment, one or more radiopaque filaments are located within the sleeve. One such filament 224 is shown by dotted lines. As with the embodiment shown in FIG. 2, the radiopaque filament is longer than the distance between the lashings 222, so that it will not be load bearing in ordinary use. Alternatively, the radiopaque filament may be secured at intervals along its length in a fashion similar to that shown in FIG. 2, and kept loose between attachment points.

FIG. 5 shows the surgical mesh device of FIG. 4 in use as a replacement for a cruciate ligament in a knee.

In FIG. 5, the eye 218 of the surgical mesh 210 is shown fitted round an anchor member 230, secured in a hole 232 in a tibia 234, and the surgical mesh 210 extends through a tunnel 236 in the tibia, then across the femur 238. The second loop 216 of the mesh is secured to another anchor member 240 secured in a hole 242 in the femur. As mentioned above, the radiopaque filament 224 is contained within the sleeve. Its length will be such that during normal use within the joint, it will not be load bearing, but will otherwise function as described above and so will break with or immediately after the surgical mesh.

FIGS. 6a and 6b shows either side of a surgical mesh device according to a further embodiment of the invention which additionally contains a stress indicator. FIG. 6c shows an exploded cross-section through part of the surgical mesh when viewed along 6c in FIG. 6a.

As with the surgical mesh shown in FIG. 2, the surgical mesh 310 of this embodiment has an eye 312, 314 at each end 316, 318. Radiopaque filaments 322 are located on the side of the mesh which is shown in FIG. 6b. The radiopaque filaments comprise a number of discrete lengths of radiopaque material anchored to the surgical mesh at points 324.

At each of a number of attachment points there is a larger anchor point 320. This may comprise radiopaque filaments or thread. The attachment points are set in a regular, predetermined pattern on the surgical mesh. Here two rows of four anchor points are shown which are spaced over a distance of a few millimetres.

In this embodiment, the radiopaque markers traverse the mesh and so are visible on both sides. However, the radiopaque markers could be physically located only on one side, for example on the same side as the radiopaque filaments.

FIG. 7 shows the surgical mesh device of FIG. 6 in situ in a shoulder to assist in the repair of an acromioclavicular joint dislocation. As with the surgical mesh in FIG. 3, during the operation to reduce the clavicle 330, the surgical mesh 310 is wrapped around the coracoid bone 332. One eye 314 is passed through the second eye 312 to secure one end of the mesh to the coracoid 332. The surgical mesh is then threaded under and over the clavicle 330 and attached to the clavicle at a fastening means, such as a screw 334.

As can be seen from FIG. 7, the radiopaque markers 320 are positioned on the surgical mesh so that, once the surgical mesh has been inserted, they will be located on the portion of the mesh which links the coracoid and clavicle. This ensures that, as the patient moves, the radiopaque markers will not rub against either bone or cause wear on the other fibres in the surgical mesh.

Should the surgical mesh device stretch, the distance between the markers will increase and hence the overall length of this section of the mesh will also increase. This will then be visible from an X-ray, when a new image is compared with one taken during or immediately after implant.

This assists medical staff in further understanding likely causes of pain in the patient and will help to predict whether the surgical mesh may be likely to rupture, before the point of failure.

In performing an operation, such as the repair of an acromioclavicular joint dislocation, a surgeon will use a kit. Such a kit will typically include a number of surgical mesh devices of different lengths and anchor means, such as screws, to attach the mesh device to bone.

The kit will often include a measuring device, so that the surgical mesh device having the most appropriate length for the patient can be selected. The kit may also include specialist tools designed for the particular operation.

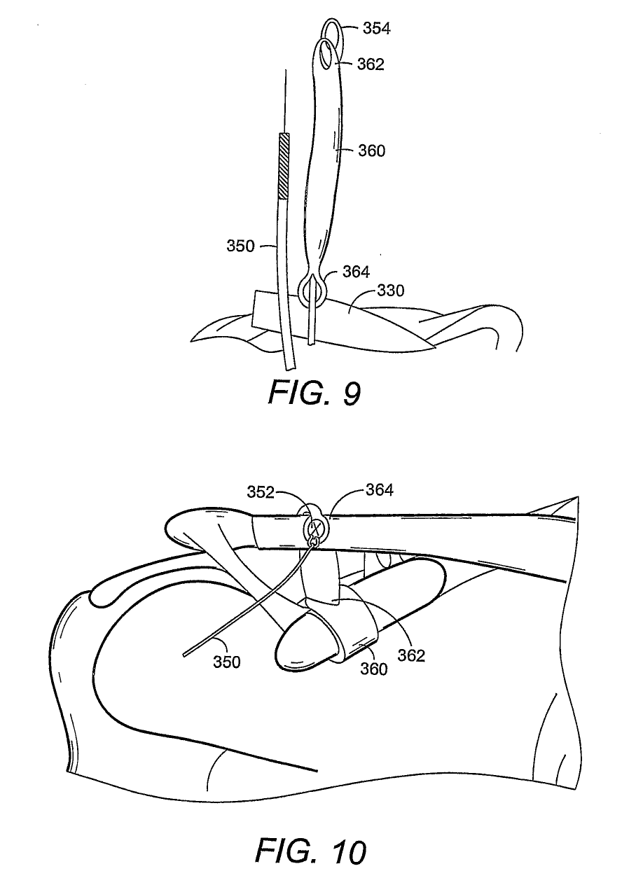

In the case of the repair of an acromioclavicular joint dislocation, the first stage (illustrated in FIG. 8) will be to wrap the measuring device 350 around the coracoid 332 and measure the length of surgical mesh required to bridge the distance when the clavicle 330 is in its reduced position between the coracoid and the intended point of attachment 352 on the clavicle 330. The measuring device can then be released and, as shown in FIG. 9, attached to the surgical mesh device by passing an eye 354 in the measuring device though the eye 364 of the surgical mesh device 360, and then passing the surgical mesh device though eye 354 in the measuring device. (For clarity, the radiopaque markers are not shown in FIGS. 9 and 10, but any of the arrangements discussed above may be used). Thus linked together, the measuring device can be used to pull the surgical mesh device into position around the coracoid. The measuring device can be used to manipulate the surgical mesh device, including assisting the threading of one eye 364 through the other eye 362 in order to secure the surgical mesh device around the coracoid. The measuring device can also assist in locating the other end of the surgical mesh device around the clavicle holding it in the right location for the intended point of attachment 352 while it is secured by the anchoring member.

The skilled person will readily appreciate alternative arrangements. One example would be an “S” shaped attachment of a surgical mesh device to two bones lying in different planes, rather than the “C” shaped attachment of FIG. 7. In this embodiment the surgical mesh device 410 is looped around a first bone 412 and then over a second bone 414 to which it is then attached.

Radiopaque material 416 is anchored to the surgical mesh device on one side at points 418 for a first part of the length of the device. For a second part of the length of the device, radiopaque material 420 is anchored to the second side of the surgical mesh device at points 422.

1. A surgical mesh device comprising an elongate length of load bearing fibres and additionally comprising non-load bearing radiopaque material anchored at more than one location along the length of the mesh, the radiopaque material enabling in situ imaging of the surgical mesh immediately following surgery during which it has been implanted and imaging of subsequent changes in the surgical mesh over time.

2. A surgical mesh device as claimed in claim 1 and wherein said radiopaque material runs along at least part of the length of the surgical mesh device, such radiopaque material being anchored to the mesh at two or more points along the length axis of the mesh, and the length of radiopaque material between such anchor points being longer in length than the distance of mesh between which the radiopaque material is anchored and wherein the length of radiopaque material between the or each pair of anchor points is sufficiently greater than the distance between the anchor points that, immediately following insertion, the radiopaque material will be non-load bearing, but whereby at or immediately following any rupture of the surgical mesh device, it will become load bearing and will itself break.

3. A surgical mesh device as claimed in claim 2 where the radiopaque material runs along the full length of the surgical mesh device.

4. A surgical mesh device as claimed in claim 2 or claim 3 where the radiopaque material is anchored at intervals along the length of the surgical mesh device.

5. A surgical mesh device as claimed in any of claims 2 to 4 wherein multiple discrete lengths of radiopaque material are anchored to the surgical mesh device.

6. A surgical mesh device as claimed in any preceding claim wherein radiopaque material is anchored to the surgical mesh device in discrete locations arranged at predetermined distances along at least part of the length of the surgical mesh device.

7. A surgical mesh device as claimed in any preceding claim wherein the surgical mesh device has a first side and a second side, the first side being intended to be in contact with bones within the patient and the second side being intended not to be in contact with the bones of the patient and the radiopaque material being anchored to the second side.

8. A surgical mesh device as claimed in any of claims 1 to 6 wherein the surgical mesh device has a first side and a second side and intended for insertion where at a first end of the surgical mesh part of the first side will contact bones within the patient and at the second end of the surgical mesh part of the second side will contact bones within the patient and along the first end the radiopaque material is anchored to the second side and along the second end the radiopaque material is anchored to the first side.

9. A surgical mesh device as claimed in any of claims 1 to 6 wherein the radiopaque material is located at a section of the surgical mesh, intermediate its length which does not contact bone.

10. A surgical mesh device as claimed in any of claims 1 to 6 wherein the surgical mesh device comprises a sleeve and the radiopaque material is contained within the sleeve.

11. A surgical mesh device as claimed in any preceding claim wherein said radiopaque material is selected from the group consisting of tungsten carbide, tungsten boride, the metals platinum, tantalum, iridium, tungsten, rhenium titanium, silver and gold, alloys of said metals, high density polymers and combinations thereof.

12. A surgical mesh device as claimed in claim 11 where the radiopaque material is selected from titanium alloy filaments, tantalum alloy filaments and polypropylene yarn loaded with a high level of barium sulphate.

13. A surgical mesh device as claimed in any preceding claim wherein the radiopaque material constitutes between 0.5% and 2% by volume of the total volume of the device.

14. A surgical mesh device as claimed in any preceding claim wherein the radiopaque material has a lower tensile strength than the surgical mesh.

15. A surgical mesh device according to any preceding claim wherein the mesh comprises a plurality of tows of biocompatible, or bioabsorbable material secured side by side in a flat elongate array and being looped to form an eye at at least one end of the device with the tows grouped together round the eye.

16. A surgical mesh device comprising a plurality of tows of biocompatible, or bioabsorbable material secured side by side in a flat elongate array and being looped to form an eye at at least one end of the device with the tows grouped together round the eye.

17. A surgical mesh device according to claim 15 or claim 16 comprising a second eye at the other end of the device.

18. A surgical mesh device according to any of claim 15 or 17 wherein at least one eye further comprises whipping around the tows.

19. A surgical mesh device according to any of claims 15 to 18 where non-load bearing radiopaque material is anchored to the mesh at or close to one or both eyes.

20. A surgical mesh device according to any preceding claim in the form of a sleeve.

21. A surgical mesh device according to claim 20 wherein the sleeve also comprises non-load bearing radiopaque material anchored at two or more points inside the sleeve, the length of radiopaque material between the or each pair of anchor points being longer in length than the distance between the points to which the radiopaque material is anchored.

22. An artificial ligament or ligament augmentation device comprising a surgical mesh device according to any preceding claim.

23. An artificial ligament or ligament augmentation device according to claim 22 which includes an autologous graft.

24. A surgical kit comprising a surgical mesh device according to any one of the preceding claims together with one or more anchor members.

25. A surgical kit according to claim 24 further comprising a set of surgical mesh devices of varying lengths, a measuring device and one or more tools for inserting and securing the surgical mesh device.

26. A method of making a surgical mesh device according to any of claims 1 to 21, comprising anchoring a length of radiopaque material to at least part of a surgical mesh, the length of radiopaque material being anchored to the surgical mesh at at least two locations along the length of the mesh and the amount of radiopaque material between points of anchoring being longer in length than the distance between which it is anchored.

27. A method of implanting a surgical mesh device according to any of claims 1 to 21 comprising the steps of securing one end of the surgical mesh device to a bone, extending the surgical mesh device across the surgery site and securing the other end of the surgical mesh device to another bone or soft tissue.

28. A method of assisting in the repair of an acromioclavicular joint dislocation comprising measuring the distance from the coracoid to the clavical in its reduced position, selecting a surgical mesh device according to any of claims 1 to 21 which mesh has a length substantially the same as the measured length, looping the mesh around the coracoid, passing a first eye through the second eye to secure a first end in place on the coracoid, attaching an anchor member to the clavicle and engaging the eye at the second end of the mesh to said anchor member,

29. A method of detecting changes in a surgical mesh device comprising inserting a surgical mesh device according to claim 6 and any of claims 7 to 21 as dependent on claim 6, imaging the operative site post operation, re-imaging the site following the patient representing with pain and comparing the images.

30. A method of detecting changes in a surgical mesh according to claim 29 wherein the insertion of the surgical mesh includes the step(s) of securing the mesh to an anchor member at one or both ends.

31. A method of detecting changes in a surgical mesh according to claim 29 wherein the surgical mesh device has an eye proximate each end and the insertion of the surgical mesh includes the step(s) of securing the mesh in the shoulder by looping it around the coracoid bone and passing the first eye through the second eye to secure a first end in place and securing the second end to an anchor member.

32. A method of detecting rupture in a surgical mesh device, including the steps of inserting a surgical mesh according to any of claims 1 to 21 in a patient and imaging the site when the patient represents with pain and reviewing the image.
